(19) United States
(12) Patent Application Publication
(10) Pub. No.: US 2017/0189344 A1
(43) Pub. Date: Jul. 6, 2017

(54) HIGHLY DRUG-LOADED POLY(ALKYL 2-CYANOACRYLATE) NANOCAPSULES

(71) Applicant: AbbVie Deutschland GmbH & Co. KG, Wiesbaden (DE)

(72) Inventors: Anamarija CURIC, Ludwigshafen (DE); Jan-Peter MÖSCHWITZER, Ludwigshafen (DE); Regina REUL, Ludwigshafen (DE)

(21) Appl. No.: 15/315,283

(22) PCT Filed: May 29, 2015
(86) PCT No.: PCT/EP2015/061933

§ 371 Date: Nov. 30, 2016

Related U.S. Application Data
(60) Provisional application No. 62/005,182, filed on May 30, 2014.

(30) Foreign Application Priority Data
May 30, 2014 (EP) 14170536.8

(51) Int. Cl. A61K 9/51; A61K 31/513; A61K 47/28; A61K 31/496

(52) U.S. Cl. CPC A61K 9/5138; A61K 31/496; A61K 31/513; A61K 47/28

(57) ABSTRACT

The present invention relates to nanocapsules which are stabilized by a bile acid or salt thereof. The nanocapsules comprise a polymeric shell formed by poly(alkyl cyanoacrylates) and/or alkoxy derivatives thereof, wherein the polymeric shell encapsulates a core comprising an active agent. The invention further relates to methods for preparing and compositions comprising such nanocapsules.

The present invention relates to nanocapsules comprising a polymeric shell encapsulating an active agent which are stabilized by a bile acid or salt thereof. The invention further relates to methods for preparing and compositions comprising such nanocapsules.

### BACKGROUND OF THE INVENTION

Nanoparticles have been studied as drug delivery systems and in particular as possible sustained release systems for targeting drugs to specific sites of action within the patient. The term “nanoparticles” is generally used to designate polymer-based particles having a diameter in the nanometer range. Nanoparticles include particles of different structure, such as nanospheres and nanocapsules, and have be described to be suspended in liquid media (e.g. aqueous or oily liquid) or a (semi-)solid phase, e.g. a polymeric phase consisting of a cellulose derivative (cf. WO 2009/073215).

Nanoparticles based on biocompatible and biodegradable polymers such as poly(alkyl cyanoacrylates) are of particular interest for biomedical applications (cf. Vauthier et al., Adv. Drug Deliv. Rev. 2003, 55:519-548). Poly(butyl cyanoacrylate) nanoparticles coated with polysorbate 80 have been shown to transport drugs which are normally unable to cross the blood-brain barrier across this barrier (Kreuter et al., Brain Res. 1995, 674:171-174; Kreuter et al., J. Drug Target. 2002, 10(4):317-325; Reimold et al., Eur. J. Pharm. Biopharm. 2008, 70:627-632).

However, a great challenge of the poly(alkyl cyanoacrylate)-based nanoparticle systems described so far is a very low drug load (Fresta et al., Biomaterials 1996, 17:751-758; Layre et al., J. Biomed. Mater. Res. 2006, Part B: Appl. Biomater. 796:254-262; Radwan 2001, J. Microencapsulation 2001, 18(4):467-477). Nanoparticles prepared by emulsion solvent evaporation methods are described to yield nanoparticles containing high amounts of polymer (often more than 80 wt-%) and, accordingly, only a low drug load (often less than 20 wt-%). Wischke et al. describes highly drug-loaded poly(butyl cyanoacrylate) capsules which, however, have diameters in the micrometer range and are instable (i.e. rupture easily) due to the high brittleness of the polymer (Int. J. Artif. Organs 2011, 34(2):243-248).

### SUMMARY OF THE INVENTION

Surprisingly, it has now been found that stable and highly drug-loaded nanocapsules can be prepared from, optionally alkoxylated, poly(alkyl cyanoacrylates) in the presence of a stabilizing agent selected from bile acids and/or bile salts.

Thus, the invention provides a nanocapsule comprising:
a) one or more than one polymer forming a polymeric shell, the polymer(s) comprising a main monomeric constituent selected from one or more than one of C₁-C₁₀-alkyl cyanoacrylates and C₁-C₆-alkoxy-C₁-C₁₀-alkyl cyanoacrylates;
b) one or more than one pharmaceutically or cosmetically active agent comprised in a core encapsulated by said polymeric shell; and
c) a nanoparticle stabilizing agent selected from one or more than one bile acid, one or more than one bile salt, and mixtures thereof.

The invention further provides a plurality of nanocapsules as described herein comprising a population of nanocapsules having a diameter of less than 500 nm, wherein the nanocapsules of the population comprise at least 50 wt-%, in particular at least 60 wt-%, at least 70 wt-%, preferably at least 80 wt-%, more preferably at least 90 wt-%, at least 95 wt-% and most preferably at least 99 wt-% or at least 99.9 wt-%, of the active agent(s) (b) relative to the total weight of shell-forming polymer(s) (a) and active agent(s) (b) of the population.

The invention also provides a method for preparing nanocapsules, the method comprising:
i) providing a hydrophobic liquid phase comprising:
   one or more than one shell-forming polymer comprising a main monomeric constituent selected from one or more than one of C₁-C₁₀-alkyl cyanoacrylates and C₁-C₆-alkoxy-C₁-C₁₀-alkyl cyanoacrylates, and
   one or more than one pharmaceutically or cosmetically active agent dissolved in a non-water-miscible organic solvent or a mixture of two or more non-water-miscible organic solvents, and
   optionally, one or more than one sorbitan fatty acid ester, and
   optionally, one or more than one amphiphilic lipid carrying a detectable moiety, a targeting moiety or a linker moiety;
ii) providing a hydrophilic liquid phase comprising:
   a nanoparticle stabilizing agent selected from one or more than one bile acid, one or more than one bile salt, and mixtures thereof
   dissolved in a hydrophilic solvent, and
   optionally, one or more than one uptake mediator selected from polyoxyethylene sorbitan fatty acid esters;
iii) finely dispersing the hydrophobic liquid phase in the hydrophilic liquid phase so as to form an emulsion; and
iv) removing at least part of the organic solvent(s) from the homogenized mixture so as to obtain a suspension of nanocapsules in the hydrophilic solvent.

The invention also provides a pharmaceutical composition comprising a plurality of nanocapsules as described herein and a pharmaceutically acceptable carrier.

### BRIEF DESCRIPTION OF THE FIGURES

FIG. 1 shows suspensions of PBCA nanoparticles prepared from different polymer-drug ratios as described in example 3 (samples 3#1 to 3#9) as well as the average particles sizes (Z-average diameters, columns) and polydispersities (PDI, curve) of these nanoparticles which were determined using a Zetasizer device.

FIG. 2 shows the zeta potentials (ZP) of PBCA nanoparticles prepared from different polymer-drug ratios as described in example 3 (samples 3#1 to 3#9) which were determined using a Zetasizer device and indicate a switch between two systems, highly-drug loaded nanocapsules and nanospheres, between polymer-drug ratios of 50:50 and 90:10.

FIG. 3 shows transmission election microscopy (TEM) images of PBCA nanoparticles prepared from different polymer-drug ratios as described in example 3 (samples 3#1 to 3#9). The reference bars indicate a length of 100 nm.

FIG. 4 shows the encapsulation efficiency (EE) of PBCA nanoparticles prepared from different polymer-drug ratios as described in example 3 (samples 3#1 to 3#9).

FIG. 5 shows the absolute drug load (AL) of PBCA nanoparticles prepared from different polymer-drug ratios as described in example 3 (samples 3#1 to 3#9).

FIG. 6 shows an overlay of Fourier Transform Infrared (FTIR) spectroscopy analysis spectra of pure crystalline Itraconazole (“ITZ pure”, light grey) and amorphous Itraconazole (“ITZ amorphous”, dark grey). Amorphous Itraconazole is characterized by a band between 1700-1800 cm⁻¹ (1), while pure crystalline Itraconazole is characterized by a band at 1000-950 cm⁻¹ (2) and a band at 900 cm⁻¹ (3).

FIG. 7 shows Fourier Transform Infrared (FTIR) spectra of PBCA nanoparticles prepared from different polymer-drug ratios as described in example 4 (samples 4#1 to 4#13) and the FITR spectra of pure crystalline Itraconazole and pure PBCA.

### DETAILED DESCRIPTION OF THE INVENTION

Nanocapsules are particles having a diameter within the nanometer range (i.e. between several nanometers to several hundred nanometers) which have a core-shell structure, i.e. a core containing the cargo (active ingredient) that is surrounded by an outer polymer layer. The nanocapsules of the invention can have a size of less than 500 nm, less than 300 nm and in particular less than 200 nm, such as in the range of from 1-500 nm, 10-300 nm or, preferably, in the range of from 50-200 nm.

Unless indicated otherwise, the terms “size” and “diameter”, when referring to a basically round object such as a nanoparticle (e.g. nanocapsules or nanospheres) or a droplet of liquid, are used interchangeably.

Size and polydispersity index (PDI) of a nanoparticle preparation can be determined, for example, by Photon Correlation Spectroscopy (PCS) and cumulant analysis according to the International Standard on Dynamic Light Scattering ISO13321 (1996) and ISO22412 (2008) which yields an average diameter (z-average diameter) and an estimate of the width of the distribution (PDI), e.g. using a Zetasizer device (Malvern Instruments, Germany).

The term “about” is understood by persons of ordinary skill in the art in the context in which it is used herein. In particular, “about” is meant to refer to variations of ±20%, ±10%, preferably ±5%, more preferably ±1%, and still more preferably ±0.1%.

The shell of the nanocapsules of the invention is formed by one or more than one polymer. The main monomeric constituent of the shell-forming polymer(s) is selected from one or more than one of C₁-C₁₀-alkyl cyanoacrylates, such as C₁-C₈-alkyl cyanoacrylates, and C₁-C₆-alkoxy-C₁-C₁₀-alkyl cyanoacrylates, such as C₁-C₃-alkoxy-C₁-C₃-alkyl cyanoacrylates. For example, the main monomeric constituent of the shell-forming polymers is selected from one or more than one of methyl 2-cyanoacrylate, 2-methoxyethyl 2-cyanoacrylate, ethyl 2-cyanoacrylate, n-butyl 2-cyanoacrylate, 2-octyl 2-cyanoacrylate and isobutyl 2-cyanoacrylate, preferably from ethyl 2-cyanoacrylate and n-butyl 2-cyanoacrylate.

The term “main monomeric constituent”, as used herein for characterizing a polymer, designates a monomeric constituent that makes up at least 80 wt-%, at least 90 wt-%, at least 95 wt-%, at least 98 wt-%, preferably at least 99 wt-% and up to 100 wt-% of the polymer.

Suitable polymers forming the shell of the nanocapsule of the invention include, but are not limited to, poly(methyl 2-cyanoacrylates), poly(2-methoxyethyl 2-cyanoacrylates), poly(ethyl 2-cyanoacrylates), poly(n-butyl 2-cyanoacrylate), poly(2-octyl 2-cyanoacrylate), poly(isobutyl 2-cyanoacrylates) and mixtures thereof, poly(n-butyl 2-cyanoacrylates), poly(ethyl 2-cyanoacrylates) and mixtures thereof being preferred.

The weight average molecular weight of the shell-forming polymers is typically in the range of from 1,000 to 10,000,000 g/mol, e.g. from 5,000 to 5,000,000 g/mol or from 10,000 to 1,000,000 g/mol.

The nanocapsules of the invention are stable (not prone to rupture). Nonetheless, they may comprise only a small amount of the polymer(s), such as less than 50 wt-%, less than 40 wt-%, less than 30 wt-%, preferably less than 20 wt-%, more preferably less than 10 wt-%, less than 5 wt-%, most preferably less than 1 wt-% or even less than 0.1 wt-% polymer(s) relative to the total weight of shell-forming polymer(s) and active agent(s) of the nanocapsule.

The shell-forming polymers described herein can be prepared by methods known in the art. In particular, they can be obtained by anionic or zwitterionic polymerization as described by, e.g., Vauthier et al. (Adv. Drug Deliv. Rev. 2003, 55:519-548) and Layre et al. (J. Biomed. Mater. Res. 2006, Part B: Appl. Biomater. 796:254-262) and the references cited therein.

The nanocapsules of the invention are preferably prepared from pre-synthesized and, if required, purified shell-forming polymer(s). The nanocapsules are therefore basically free of residual monomers of the shell-forming polymer(s) such as C₁-C₁₀-alkyl cyanoacrylates, C₁-C₆-alkoxy-C₁-C₁₀-alkyl cyanoacrylates, and salts of these acids.

“Basically free of residual monomers” refers to amounts of less than 10 wt-%, preferably less than 5 wt-%, more preferably less than 2 wt-% and in particular less than 1 wt-%, for example less than 0.01 wt-% or less than 0.05 wt-%, monomer relative to the total weight of shell-forming polymer(s).

Typically, polymerization is performed in an aqueous medium or, preferably, water under agitation (e.g. stirring). For preparing nanocapsules according to the invention the polymer is typically applied in the form of a powder. Such polymer powder can be obtained by freeze drying the aqueous polymer suspension obtained after polymerization. Agglomerates are expediently removed from the polymer suspension; they can converted into polymer powder by diluting the agglomerates in a water-miscible organic solvent such as acetone, adding an excess of water to the organic solution to precipitate the polymer, evaporating the organic solvent and freeze drying the aqueous polymer suspension.

The nanocapsules of the invention are suitable for the delivery of cargo molecules such as pharmaceutically or cosmetically active agents and nutritional supplements (herein also generally referred to as “active agents”).

The invention is particularly useful for the encapsulation and targeted delivery of water-insoluble or poorly water-soluble (or “lipophilic”) compounds. Compounds are considered water-insoluble or poorly water-soluble if their solubility in water at 25° C. (at pH 7.0) is 1 g/100 ml or less. In particular, the active agent encapsulated according to the invention has solubility in water at 25° C. (at pH 7.0) of 0.1 g/100 ml or less, 0.05 g/100 ml or less, preferably 0.01 g/100 ml or less 0.005 g/100 ml or less, or most preferably of 0.001 g/100 ml or less.

The nanocapsules of the invention protect the cargo molecules on the way to the target site (e.g. the target cell) from degradation and/or modification by proteolytic and other enzymes and thus from the loss of their biological (e.g. pharmaceutical, cosmetical or nutritional) activity. The invention is therefore also particularly useful for encapsulating molecules which are susceptible to such enzymatic degradation and/or modification, especially if administered by the oral route.

The active agents encapsulated in nanocapsules of the invention typically have molecular weights of less than 2000 g/mol, in particular a molecular weight in the range of from 100-2000 g/mol.

The active agents encapsulated in nanocapsules of the invention typically belong to classes 2 or 4 of the Biopharmaceutics Classification System (BCS, as provided by the U.S. Food and Drug Administration), which both represent agents with low solubility.

Specific examples of pharmaceutically active agents according to the invention include, but are not limited to:

(2R,4S)-rel-1-(butan-2-yl)-4-{4-[4-(4-{[(2R,4S)-2-(2,4-dichlorophenyl)-2-(1H-1,2,4-triazol-1-ylnnethyl)-1,3-dioxolan-4-yl]methoxy}phenyl)piperazin-1-yl]phenyl}-4,5-dihydro-1H-1,2,4-triazol-5-one [Itraconazole]; (2S)—N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide [Lopinavir]; Aceclofenac, Albendazole, Amiodarone, Amphotericin B, Aquinavir, Atorvastatin, Atovaquone, Azithrom, Carbamazepine, Carvedilol, Chlorothiazide, Chlorpromazine, Chlorthalidone, Ciprofloxacin, Cisapride, Colistin, Cyclosporine, Danazole, Dapsone, Diclofenac, Diflunisal, Digoxin, Erythromycin, Flurbiprofen, Furosemide, Glipizide, Glyburide, Griseofulvin, Hydrochlorothiazide, Ibuprofen, Indinavir, Indomethacin, Ketoconazole, Lansoprazolel, Lovastatin, Mebendazole, Methotrexate, Miconazole, Nelfinavir, Neomycin, Nevirapine, Ofloxacin, Oxaprozin, Oxaprozin, Phenazopyridine, Phenytoin, Pilocarpine, Piroxicam, Raloxifene, Ritonavir, salicylic acid, Sirolimus, Spironolactone, Tacrolimus, Talinolol, Tamoxifen, Terfenadine, Troglitazone and Valtrasan.

The core of the nanocapsules of the invention comprises the active agent(s) described herein. Although the active agent(s) may be liquid or in the form of a liquid (e.g. aqueous or oily) solution or dispersion, this is generally not preferred. Rather, according to one embodiment, at least 50%, in particular at least 70%, at least 80%, at least 90% or, preferably at least 95% or 100% of the active agent(s) or nutritional supplement(s) in the nanocapsule core is/are present in an undissolved solid form, such as an amorphous, semi-crystalline or crystalline state, or a mixture thereof.

According to a particular embodiment, at least 50%, in particular at least 70%, at least 80%, at least 90% or, preferably at least 95% or 100% of the active agent(s) or nutritional supplement(s) in the nanocapsule core is/are present in a crystalline state.

According to an even another particular embodiment, at least 50%, in particular at least 70%, at least 80%, at least 90% or, preferably at least 95% or 100% of the active agent(s) or nutritional supplement(s) in the nanocapsule core is/are present in a semi-crystalline state.

According to another particular embodiment, at least 50%, in particular at least 70%, at least 80%, at least 90% or, preferably at least 95% or 100% of the active agent(s) or nutritional supplement(s) in the nanocapsule core is/are present in an amorphous state.

The invention provides nanocapsules which may have an advantageously high load of cargo molecules. Thus, the nanocapsule of the invention can comprise at least 50 wt-%, at least 60 wt-%, at least 70 wt-%, preferably at least 80 wt-%, more preferably at least 90 wt-%, at least 95 wt-%, most preferably at least 99 wt-% or at least 99.9 wt-% and up to 99.9 wt-%, up to 99.95 wt-% or, preferably, up to 99.99 wt-% of the active agent(s) relative to the total weight of shell-forming polymer(s) and active agent(s) of the nanocapsule.

The invention further provides a plurality of nanocapsules as described herein, wherein nanocapsules having a diameter of less than 500 nm have an advantageously high load of cargo molecules and may be present in an advantageously high proportion.

Thus, the invention provides a plurality of nanocapsules comprising a population of nanocapsules having a diameter of less than 500 nm, less than 300 nm or, preferably, less than 200 nm (such as in the range of from 1-500 nm, 10-300 nm or, preferably, in the range of from 50-200 nm), wherein the nanocapsules of the population comprise at least 50 wt-%, at least 60 wt-%, at least 70 wt-%, preferably at least 80 wt-%, more preferably at least 90 wt-%, at least 95 wt-%, most preferably at least 99 wt-% or at least 99.9 wt-% and up to 99.9 wt-%, up to 99.95 wt-% or, preferably, up to 99.99 wt-% of the active agent(s) relative to the total weight of shell-forming polymer(s) and active agent(s) of the population. The plurality of nanocapsules according to the invention can comprise a population of nanocapsules having a diameter of less than 500 nm, less than 300 nm or, preferably, less than 200 nm, such as in the range of from 1-500 nm, 10-300 nm or, preferably, in the range of from 50-200 nm, wherein this population accounts for more than 90 wt-% of the plurality of nanocapsules.

The term “plurality of nanocapsules” refers to 2 or more nanocapsules, for example at least 10, at least 100, at least 1,000, at least 5,000, at least 10,000, at least 50,000, at least 100,000, at least 500,000, or at least 1,000,000 or more nanocapsules.

The nanocapsules of the invention comprise a nanoparticle stabilizing agent selected from one or more than one bile acid, one or more than one bile salt, and mixtures thereof. The nanoparticle stabilizing agent allows for the formation of stable nanocapsules even where the nanocapsules are highly drug-loaded, i.e. the amount of shell-forming polymer is very low.

Examples of suitable nanoparticle stabilizing agents include bile acids, such as cholic acid, taurocholic acid, glycocholic acid, deoxycholic acid, lithocholic acid, chenodeoxycholic acid, dehydrocholic acid, ursodeoxycholic acid, hyodeoxycholic acid, hyocholic acid, and mixtures thereof, as well as salts (e.g. sodium, potassium or calcium salts) of said acids, and mixtures thereof. Preferably, the nanoparticle stabilizing agent is selected from cholic acid, one or more than one salt of cholic acid, and mixtures thereof. According to a most preferred embodiment, the nanoparticle stabilizing agent is sodium cholate.

The one or more than one nanoparticle stabilizing agent is typically present in an amount of from 3 to 36 wt-% relative to the total weight of shell-forming polymer(s) and active agent(s) of the nanocapsule.

Optionally, the nanocapsule of the invention may further comprise one or more than one uptake mediator selected from polyoxyethylene sorbitan fatty acid esters. Said uptake mediator(s) can facilitate the transport of the nanocapsules across barriers within the organism, in particular across the blood-brain barrier. It is hypothesized that polyoxyethylene sorbitan fatty acid esters such as Tween 80 (polysorbate 80) facilitate an attraction of specific plasma proteins, such as ApoE, which play a key role in the receptor-mediated uptake of compounds by brain capillary cells.

Examples of uptake mediators include polyoxyethylene sorbitan monoesters and triesters with monounsaturated or, in particular, saturated fatty acids. Examples of particular fatty acids include, but are not limited to, C₁₁-C₁₈-fatty acids such as lauric acid, palmitic acid, stearic acid and, in particular, oleic acid. The polyoxyethylene sorbitan fatty acid esters may comprise up to 90 oxyethylene units, for example 15-25, 18-22 or, preferably, 20 oxyethylene units. The uptake mediator(s) is/are preferably selected from polyoxyethylene sorbitan fatty acid esters having an HLB value in the range of about 13-18, in particular about 16-17. Expediently, the uptake mediator(s) used in the nanocapsules of the invention are selected from officially approved food additives such as, for example, E432 (polysorbate 20), E434 (polysorbate 40), E435 (polysorbate 60), E436 (polysorbate 65) and, in particular, E433 (polysorbate 80). Preferably, the uptake mediator is polyoxyethylene (20) sorbitan monooleate.

The one or more than one uptake mediator is typically present in an amount of from 0.001 to 0.1 wt-% relative to the total weight of shell-forming polymer(s) and active agent(s) of the nanocapsule.

Optionally, the nanocapsule of the invention may further comprise one or more than one sorbitan fatty acid ester. Said sorbitan fatty acid(s) can facilitate the formation of nanocapsules having a reduced size, e.g. a diameter of less than 200 nm.

Examples of suitable sorbitan fatty acid esters include, but are not limited to, sorbitan monoesters of monounsaturated or, in particular, saturated C₁₁-C₁₈-fatty acids such as lauric acid, palmitic acid, stearic acid and, in particular, oleic acid. Preferably, the nanocapsule of the invention comprises sorbitan monooleate.

Optionally, the nanocapsules of the invention, and in particular the polymeric shell thereof, may include one or more than one amphiphilic lipids that, for example, can serve as a detectable label, is linked to a targeting compound or carries a linker allowing for the attachment of, for example, targeting or labelling compounds.

The term “amphiphilic lipid”, as used herein, refers to a molecule comprising a hydrophilic part and a hydrophobic part. Generally, the hydrophobic part of an amphiphilic lipid comprises one or more than one linear or branched saturated or unsaturated hydrocarbon chain having from 7 to 29 carbon atoms (i.e. is derived from a C₈-C₃₀ fatty acid). Examples of suitable amphiphilic lipids for use in the nanocapsules of the invention include naturally occurring or synthetic phospholipids, cholesterols, lysolipids, sphingomyelins, tocopherols, glucolipids, stearylamines and cardiolipins. Further examples include esters and ethers of one or more than one (e.g. one or two) fatty acid with a hydrophilic compound such as a sugar alcohol (e.g. sorbitan) or saccharide (such as a mono-, di- or trisaccharide, e.g. saccharose). The amphiphilic lipid used in the nanocapsules of the invention expediently carries a functional moiety, such as a linker, detectable and/or targeting moiety. Said moiety is preferably covalently coupled to the hydrophilic part of the amphiphilic lipid, optionally via a spacer. Such spacer may comprise, or essentially consist of, a polyoxyethylene chain.

The amphiphilic lipid used in the nanocapsules of the invention is preferably a phospholipid that carries a functional moiety selected from a linker, detectable and/or targeting moiety as described herein.

The term “lipid”, as used herein, refers to a fat, oil or substance containing esterified fatty acids present in animal fats and in plant oils. Lipids are hydrophobic or amphiphilic molecules mainly formed of carbon, hydrogen and oxygen and have a density lower than that of water. Lipids can be in a solid state at room temperature (25° C.), as in waxes, or liquid as in oils.

The term “fatty acid”, as used herein, refers to an aliphatic monocarboxylic acid having a, generally linear, saturated or unsaturated hydrocarbon chain and at least 4 carbon atoms, typically from 4 to 30 carbon atoms. Natural fatty acids mostly have an even number of carbon atoms and from 4 to 30 carbon atoms. Long chain fatty acids are those having from 14 to 22 carbon atoms; and very long chain fatty acids are those having more than 22 carbon atoms.

The term “phospholipid”, as used herein, refers to a lipid having a phosphate group, in particular a phosphoglyceride. Phospholipids comprise a hydrophilic part including the phosphate group and a hydrophobic part formed by (typically two) fatty acid hydrocarbon chains. Particular phospholipids include phosphatidylcholine, phosphatidylethanolamines (e.g. 1,2-distearoyl-sn-glycero-3-phosphoethanolamine), phosphatidylinositol, phosphatidylserine and sphingomyelin which carry a functional moiety as described herein.

According to one embodiment, the nanocapsule of the invention, and in particular the polymeric shell thereof, comprises one or more than one amphiphilic lipid, wherein said amphiphilic lipid carries a detectable moiety. Suitable detectable moieties include, but are not limited to, fluorescent moieties and moieties which can be detected by an enzymatic reaction or by specific binding of a detectable molecule (e.g. a fluorescence-labelled antibody), fluorescent moieties (such as, for example, fluorescein or rhodamine B) being preferred. According to a particular embodiment, the nanocapsule of the invention, and in particular the polymeric shell thereof, comprises one or more than one phospholipid (e.g. phosphatidylethanolamine) carrying a fluorescent moiety. Typically, the amount of amphiphilic lipid(s) comprising a detectable moiety, and in particular a fluorescent moiety, is in the range of 0.01-2 wt-%, in particular 0.1-1.5 wt-%, and preferably 0.5-1 wt-%, relative to the total weight of shell-forming polymer(s) (a) and active agent(s) (b) of the nanocapsule.

According to a further embodiment, the nanocapsule of the invention, and in particular the polymeric shell thereof, comprises one or more than one amphiphilic lipid, wherein said amphiphilic lipid, and preferably the hydrophilic part thereof, carries a targeting moiety. Targeting moieties are capable of binding specifically to a target molecule (e.g. a cell surface molecule characteristic for a particular type of cells), which allows nanocapsules comprising amphiphilic lipids with such target moieties to accumulate at a particular target site (e.g. in a particular organ or tissue) within a subjects body. Suitable targeting moieties include, but are not limited to, antibodies (such as conventional and single-domain antibodies), antigen-binding fragments and derivatives thereof, as well as ligands and ligand analogues of cell surface receptors. Typically, the amount of amphiphilic lipid(s) comprising a targeting moiety, and in particular an antibody or antigen-binding fragment thereof, is in the range of 0.01-10 wt-%, in particular 0.1-7 wt-%, and preferably 0.5-5 wt-%, relative to the total weight of shell-forming polymer(s) (a) and active agent(s) (b) of the nanocapsule.

According to a further embodiment, the nanocapsule of the invention, and in particular the polymeric shell thereof, comprises one or more than one amphiphilic lipid, wherein said amphiphilic lipid, and preferably the hydrophilic part thereof, carries a linker moiety. Linker moieties allow for the attachment of, for example, targeting and/or labelling compounds to the amphiphilic lipid, in particular via covalent coupling so as to form amphiphilic lipids comprising detectable or targeting moieties as described herein. Thus, compounds such as targeting or labeling compounds can be attached (e.g. coupled covalently) to the surface of nanocapsules comprising (incorporated in their polymeric shell) one or more than one amphiphilic lipid carrying a linker moiety. Suitable linker moieties have a reactive function, such as a maleimide, carboxy, succinyl, azido, 2-pyridyldithio, 2,4-dichlorotriazinyl, sulfhydryl, amino, biotinyl or aldehyde group, with maleimide being preferred. Typically, the amount of amphiphilic lipid(s) comprising a linker moiety is in the range of 0.01-10 wt-%, in particular 0.1-7 wt-%, and preferably 0.5-5 wt-%, relative to the total weight of shell-forming polymer(s) (a) and active agent(s) (b) of the nanocapsule.

Further suitable agents which can be coupled to the amphiphilic lipid used in nanocapsules of the invention (as described for detectable and targeting compounds herein) include compounds which are capable of making the nanocapsules invisible to the immune system (such as folic acid), increase the circulation time of the nanocapsules within the subject and/or slow down elimination of the nanocapsules.

The nanocapsule of the invention may comprise more than one type of amphiphilic lipid described herein, thus combining different functions such as targeting and labeling on one and the same nanocapsule.

The components of the nanocapsules of the invention, in particular the shell-forming polymer(s), as well as the ingredients of compositions according to the invention, in particular the carrier, are, expediently, pharmaceutically acceptable.

The term “pharmaceutically acceptable”, as used herein, refers to a compound or material that does not cause acute toxicity when nanocapsules of the invention or a composition thereof is administered in the amount required for medical or cosmetic treatment or medical prophylaxis, or that is taken up by consumption of the maximum recommended intake of a nutritional product comprising nanocapsules of the invention or a composition thereof.

The nanocapsules of the invention can be prepared by an emulsion solvent evaporation method, in particular by a method comprising:
i) providing a hydrophobic liquid phase comprising:
   one or more than one shell-forming polymer comprising a main monomeric constituent selected from one or more than one of C₁-C₁₀-alkyl cyanoacrylates and C₁-C₆-alkoxy-C₁-C₁₀-alkyl cyanoacrylates, and
   one or more than one pharmaceutically or cosmetically active agent dissolved in a non-water-miscible organic solvent or a mixture of two or more non-water-miscible organic solvents, and
   optionally, one or more than one sorbitan fatty acid ester, and
   optionally, one or more than one amphiphilic lipid carrying a detectable moiety, a targeting moiety or a linker moiety;
ii) providing a hydrophilic liquid phase comprising:
   a nanoparticle stabilizing agent selected from one or more than one bile acid, one or more than one bile salt, and mixtures thereof
   dissolved in a hydrophilic solvent, and
   optionally, one or more than one uptake mediator selected from polyoxyethylene sorbitan fatty acid esters;
iii) finely dispersing the hydrophobic liquid phase in the hydrophilic liquid phase so as to form an emulsion; and
iv) removing at least part of the organic solvent(s) from the homogenized mixture so as to obtain a suspension of nanocapsules in the hydrophilic solvent.

In contrast to methods such as interfacial polymerization or emulsion polymerization, the method of the invention starts with preformed (shell-forming) polymer which allows a better control of polymer properties and a reduction of the residual monomer content.

The organic solvents useful for providing the hydrophobic liquid phase in step (i) of the method of the invention are non-water-miscible solvents. The term “non-water-miscible solvents”, as used herein, refers to solvents having a solubility in water of less than about 10 wt-%, in particular less than about 5 wt-%, and preferably less than about 3 wt-%. Non-water-miscible organic solvents for use in step (i) are preferably volatile, i.e are liquid at room temperature (25° C.) and have a boiling point of 150° C. or less at standard pressure (100 kPa). Examples of suitable non-water-miscible organic solvents include, but are not limited to, chloroform, methylene chloride, trichloroethylene, trichloro-trifluoroethylene, tetrachloroethane, trichloroethane, dichloroethane, dibromoethane, ethyl acetate, phenol, toluene, xylene, ethyl-benzene, benzyl alcohol, creosol, methyl-ethyl ketone, methyl-isobutyl ketone, hexane, heptane, furan and non-cyclic aliphatic ethers such diethyl ether, as well as mixtures thereof, chloroform being preferred.

The hydrophilic solvent used for providing the hydrophilic liquid phase in step (ii) of the method of the invention is preferably water.

Emulsion solvent evaporation methods, wherein the volume of hydrophilic phase is very high relative to the volume of the hydrophobic phase, yield very dilute nanocapsule suspensions, which may require processing steps to increase the concentration of nanocapsules in the suspension to a concentration sufficiently high for the ultimate use. The volume ratio of hydrophobic liquid phase:hydrophilic liquid phase is generally in the range of from 1:100 to 2:3, preferably in the range of from 1:9 to 1:2.

The hydrophobic liquid phase is finely dispersed in the hydrophilic liquid phase so as to form an emulsion of fine droplets of the hydrophobic liquid distributed throughout the hydrophilic liquid. This emulsion may be obtained, by applying shear forces, for example by thorough mixing using a static mixer, by ultrasound, by homogenization under pressure, e.g. under a pressure of at least 5,000 kPa, such as from 20,000 to 200,000 kPa, preferably from 50,000 to 100,000 kPa, or by combining any of these homogenization methods. The emulsion of the hydrophobic liquid in the hydrophilic liquid can be prepared in a two-step process, wherein the two phases are first mixed, e.g. with a static mixer (rotator/stator-type mixer), so as to obtain a pre-emulsion which, in a second step, is further homogenized ultrasonically and/or using a high pressure homogenizer so as to reduce the size of the hydrophobic liquid droplets. The shear forces may be applied for a time of from 1-12 min, in particular from 4-10 min. For example, ultrasound may be applied for 1-10 min, in particular from 2-5 min, with amplitude in the range of from 60-100%, in particular 70-100%.

At least part of the organic solvent(s) is then removed from the homogenized mixture so as to obtain a suspension of nanocapsules in a hydrophilic, preferably aqueous, medium (comprising the hydrophilic solvent). Suitable measures for removing organic solvent from a homogenized mixture, such as in step (iv) of the method of the invention, are known in the art and include, but are not limited to, evaporation, extraction, diafiltration, pervaporation, vapor permeation and filtration. The concentration of organic solvent in the hydrophilic suspension medium of the nanocapsules is expediently reduced to below the solubility of the organic solvent in the said medium, in particular to a concentration of less than about 5 wt-%, less than about 3 wt-%, less than about 1 wt-% and preferably less than about 0.1 wt-%. Preferably, the organic solvent(s) is/are removed to an extent that the resulting suspension of nanocapsules is pharmaceutically acceptable or acceptable according to the ICH (International Committee on Harmonization) guidelines, respectively.

Optionally, the method of the invention may further comprise purification steps such as the removal of drug precipitates and agglomerates, e.g. by filtration, and/or a partial or complete exchange of the suspension medium, e.g. by dialysis.

The method of the invention can yield preparations of nanocapsules having a relatively high uniformity with respect to size, for example preparations, wherein the majority of the nanocapsules has a diameter of less than 500 nm, less than 300 nm and in particular less than 200 nm, such as in the range of from 1-500 nm, 10-300 nm or, in particular, in the range of from 50-200 nm. In particular, nanocapsule preparations obtained with the method of the invention can have PDI values as determined by Photon Correlation Spectroscopy of 0.5 or less, 0.3 or less, preferably 0.2 or less, or even 0.1 or less. Nonetheless, the nanocapsule preparation may be processed further (e.g. by filtration) to remove nanocapsules having diameters outside a desired range.

The relative amounts of shell-forming polymer(s) and active agent(s) used in the method of the invention can be as high as at least 50 wt %, at least 60 wt-%, at least 70 wt-%, preferably at least 80 wt-%, more preferably at least 90 wt-%, at least 95 wt-%, most preferably at least 99 wt-% or at least 99.9 wt-% and up to 99.9 wt %, up to 99.95 wt-% or, preferably, up to 99.99 wt-% of the active agent(s) relative to the total weight of shell-forming polymer(s) and active agent(s).

The presence of a nanoparticle stabilizing agent selected from one or more than one bile acid, one or more than one bile salt, and mixtures thereof, as described herein, allows for the formation of highly stable nanocapsules, high encapsulation efficiency as well as high absolute drug loading.

The term “encapsulation efficiency” (EE) refers to the amount of active agent(s) encapsulated in nanocapsules relative to the total amount of active agent(s) used for preparing the nanocapsules. The method of the present invention allows for encapsulation efficiencies of at least 50%, at least 60%, at least 70% or even at least 80%, using at least 50 wt-%, in particular at least 60 wt-% and preferably at least 80 wt-% active agent(s) relative to the total weight of shell-forming polymer(s) and active agent(s) used in the preparation of the nanocapsule.

The term “absolute drug loading” (AL) refers to the weight of active agent(s) encapsulated in the nanocapsule relative to the total weight of the active agent(s) plus shell-forming polymer polymer(s). Absolute drug loading is one of the most important measures considering the application dose. In contrast to previously described nanoparticles based on poly(alkyl cyanoacrylates), the nanocapsules according to the invention can have significantly increased absolute drug loadings such as at least 50 wt-%, at least 60 wt-%, or even at least 70 wt-%.

The concentration of nanoparticle stabilizing agent in the hydrophilic phase provided in step (ii) of the method of the invention is typically in the range of from 50% to 150%, in particular from 80% to 120% and preferably from 90% to 110%, of its critical micelle concentration, for example in the range of from 5 mM to 15 mM, in particular from 8 mM to 12 mM and specifically from 9 mM to 11 mM.

The term “critical micelle concentration” (CMC) refers to the concentration of a surfactant above which micelles form.

The hydrophilic liquid phase provided in step (ii) of the method of the invention can further comprise one or more than one uptake mediator selected from polyoxyethylene sorbitan fatty acid esters as described herein. Particularly suitable concentrations of the sorbitan fatty acid ester(s) in the hydrophobic liquid phase are in the range of from 50% to 150%, in particular from 80% to 120% and preferably from 90% to 110%, of its critical micelle concentration, for example in the range of from 6 μM to 18 μM, in particular from 9.6 μM to 14.4 μM and specifically from 10.8 μM to 13.2 μM.

The hydrophobic liquid phase provided in step (i) of the method of the invention can further comprise one or more than one sorbitan fatty acid ester as described herein. Particularly suitable concentrations of the sorbitan fatty acid ester(s) in the hydrophobic liquid phase are in the range of from 0.1 M to 0.2 M, specifically from 0.12 M to 0.18 M.

The hydrophobic liquid phase provided in step (i) of the method of the invention can further comprise one or more than one amphiphilic lipid as described herein.

The invention further provides a pharmaceutical composition comprising a plurality of nanocapsules as described herein, and a pharmaceutically acceptable carrier. The carrier is chosen to be suitable for the intended way of administration which can be, for example, oral or parenteral administration, intravascular, subcutaneous or, most commonly, intravenous injection, transdermal application, or topical applications such as onto the skin, nasal or buccal mucosa or the conjunctiva.

The nanocapsules of the invention can increase the bioavailability and efficacy of the encapsulated active agent(s) by protecting said agent(s) from premature degradation in the gastrointestinal tract and/or the blood, and allowing for a sustained release thereof. Following oral administration, the nanocapsules of the invention can traverse the intestinal wall and even barriers such as the blood-brain barrier.

Liquid pharmaceutical compositions of the invention typically comprise a carrier selected from aqueous solutions which may comprise one or more than one water-soluble salt and/or one or more than one water-soluble polymer. If the composition is to be administered by injection, the carrier is typically an isotonic aqueous solution (e.g. a solution containing 150 mM NaCl, 5 wt-% dextrose or both). Such carrier also typically has an appropriate (physiological) pH in the range of from about 7.3-7.4.

Solid or semisolid carriers, e.g. for compositions to be administered orally or as an depot implant, may be selected from pharmaceutically acceptable polymers including, but not limited to, homopolymers and copolymers of N-vinyl lactams (especially homopolymers and copolymers of N-vinyl pyrrolidone, e.g. polyvinylpyrrolidone, copolymers of N-vinyl pyrrolidone and vinyl acetate or vinyl propionate), cellulose esters and cellulose ethers (in particular methylcellulose and ethylcellulose, hydroxyalkylcelluloses, in particular hydroxypropylcellulose, hydroxylalkylalkyl-celluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate), high molecular weight polyalkylene oxides (such as polyethylene oxide and polypropylene oxide and copolymers of ethylene oxide and propylene oxide), polyvinyl alcohol-polyethylene glycol-graft copolymers, polyacrylates and polymethacrylates (such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates)), polyacrylamides, vinyl acetate polymers (such as copolymers of vinyl acetate and crotonic acid, partially hydrolyzed polyvinyl acetate), polyvinyl alcohol, oligo- and polysaccharides such as carrageenans, galactomannans and xanthan gum, or mixtures of one or more thereof. Solid carrier ingredients may be dissolved or suspended in a liquid suspension of nanocapsules of the invention and the liquid suspension medium may be, at least partially, removed.

### EXAMPLES

### Determination of Particle Size and Polydispersity Index

In the examples described herein, size and polydispersity index (PDI) of the prepared nanoparticles were determined by Photon Correlation Spectroscopy (PCS) and cumulant analysis according to the International Standard on Dynamic Light Scattering ISO13321 (1996) and ISO22412 (2008) using a Zetasizer device (Malvern Instruments, Germany; software version “Nano ZS”) which yields an average diameter (z-average diameter) and an estimate of the width of the distribution (PDI). The PDI, as indicated in the examples, is a dimensionless measure of the broadness of the size distribution which, in the Zetasizer software ranges from 0 to 1. PDI values of <0.05 indicate monodisperse samples (i.e. samples with a very uniform particle size distribution), while higher PDI values indicate more polydisperse samples.

### Preparation of poly(n-butyl 2-cyanoacrylate)

Polymer synthesis was performed as described by Layre et al. (J. Biomed. Mater. Res. 2006, Part B: Appl. Biomater. 796:254-262). 1 ml n-butyl 2-cyanoacrylate was slowly added to 15 ml water and the mixture was incubated for 2 h at room temperature while stirring (300 rpm). The resulting milky suspension was collected and lyophilized by freeze drying. Some agglomerates which had formed around the stirrer were diluted in acetone. The polymer was precipitated by adding a 10-fold excess of water, the acetone was evaporated from the precipitate at room temperature while stirring and the polymer was freeze dried.

### Example 1 Preparation of Itraconazole-Loaded poly(n-butyl 2-cyanoacrylate) Nanocapsules with and without Tween 80

Sample with Sodium Cholate:

1 ml of a solution of 9.5 mg/ml Itraconazole and 0.5 mg/ml poly(n-butyl 2-cyano-acrylate) (PBCA) in chloroform was added to 2 ml of an aqueous solution of 10 mM sodium cholate.

Sample with Sodium Cholate and Tween 80:

1 ml of a solution of 9.5 mg/ml Itraconazole and 0.5 mg/ml poly(n-butyl 2-cyanoacrylate) in chloroform were added to 2 ml of an aqueous solution of 10 mM sodium cholate and 10 μM Tween 80.

Each sample (in a 7 ml glass vial) was sonicated (70% amplitude, 1 cycle) for 10 min at room temperature. After transfer into a larger (20 ml) glass vial, the emulsion was stirred at room temperature until the chloroform had evaporated (monitored gravimetrically). Size and PDI of the particles in the obtained suspension were determined. The particles (prepared with or without Tween 80) were found to be uniform and smaller than 200 nm. The suspension was filtered through a 200 nm membrane to remove precipitates of non-encapsulated Itraconazole (which precipitated in aqueous environment). After filtration the size and PDI of the particles were measured again and the concentration of Itraconazole was determined by Reverse-Phase High Performance Liquid Chromatography (RP-HPLC).

### Example 2 Preparation of Itraconazole-Loaded poly(n-butyl 2-cyanoacrylate) Nanocapsules with and without Tween 80

Itraconazole-loaded PBCA nanocapsules were prepared and analyzed as described in EXAMPLE 1, except from sonicating (70%, 1 cycle) the samples for 4 min while cooling on ice. The resulting nanoparticles (prepared with or without Tween 80) had diameters in the range of approximately 500-650 nm and thus were larger than those obtained in EXAMPLE 1. These results confirm that smaller particle sizes can be obtained by more intense homogenization.

### Example 3 Preparation of Itraconazole-Loaded poly(n-butyl 2-cyanoacrylate) Nanocapsules with Different Polymer-Drug Ratios

Samples 3#1 to 3#9: For each sample, 1 ml of a solution of Itraconazole and poly(n-butyl 2-cyanoacrylate) in chloroform, having concentrations as indicated in Table 1, was added to 2 ml of an aqueous solution of 10 mM sodium cholate and 10 μM Tween 80. Each sample (in a 7 ml glass vial) was sonicated (70%, 1 cycle) for 10 min at room temperature. After transfer into a larger (20 ml) glass vial, the emulsion was stirred at room temperature until the chloroform had evaporated (monitored gravimetrically). The suspension was filtered through a 200 nm membrane to remove precipitates of non-encapsulated Itraconazole (which precipitated in aqueous environment). After filtration the size and PDI of the particles were measured.

TABLE 1
Composition of the solution of Itraconazole
and PBCA in chloroform

| | poly(n-butyl | | |
|---|---|---|---|
| Sample | 2-cyanoacrylate) | Itraconazole | polymer-drug |
| # | [mg/ml CHCl₃] | [mg/ml CHCl₃] | ratio |
| 3#1 | 0.25 | 24.75 | 1:99 |
| 3#2 | 1.25 | 23.75 | 5:95 |
| 3#3 | 2.50 | 22.50 | 10:90 |
| 3#4 | 5.00 | 20.00 | 20:80 |
| 3#5 | 12.50 | 17.50 | 50:50 |
| 3#6 | 20.00 | 5.00 | 80:20 |
| 3#7 | 22.50 | 2.50 | 90:10 |
| 3#8 | 23.75 | 1.25 | 95:5 |
| 3#9 | 24.75 | 0.25 | 99:1 |

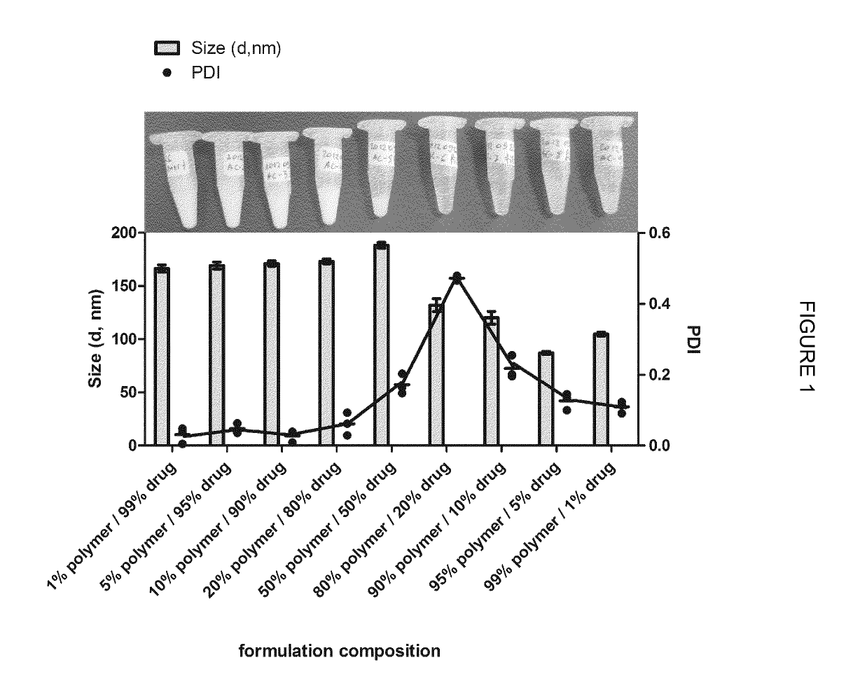

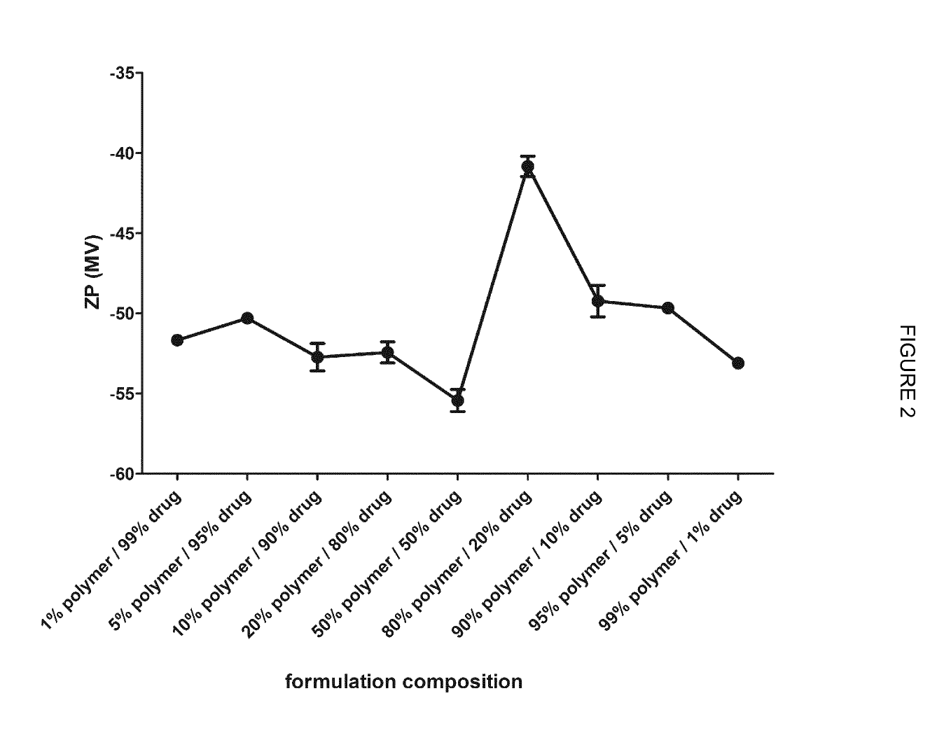

The results are summarized in FIG. 1 and indicate that there was a switch between two systems: highly-drug loaded nanocapsules having a size (Z-average diameter) of about 170-190 nm and nanospheres of about 80-140 nm containing significantly smaller drug loads. The measurement of the zeta potential (ZP) confirmed this switch between polymer-drug ratios of 50:50 and 90:10 (cf. FIG. 2).

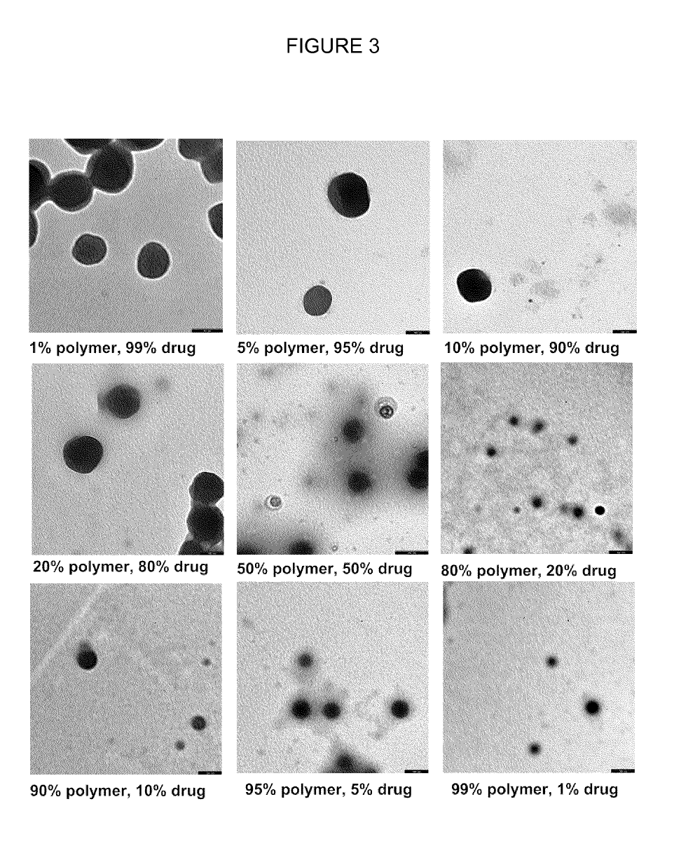

Additionally, the determined size and size distribution as well as the switch from larger nanocapsules to smaller nanospheres were confirmed by transmission election microscopy (TEM, cf. FIG. 3). As assumed, the larger particles (nanocapsules) obtained at polymer-drug ratios of 50:50 and below contained a drug core with a very thin outer polymer layer, while in the smaller particles (nanospheres) obtained at polymer-drug ratios of 80:20 the small amounts of drug were distributed in the polymer matrix.

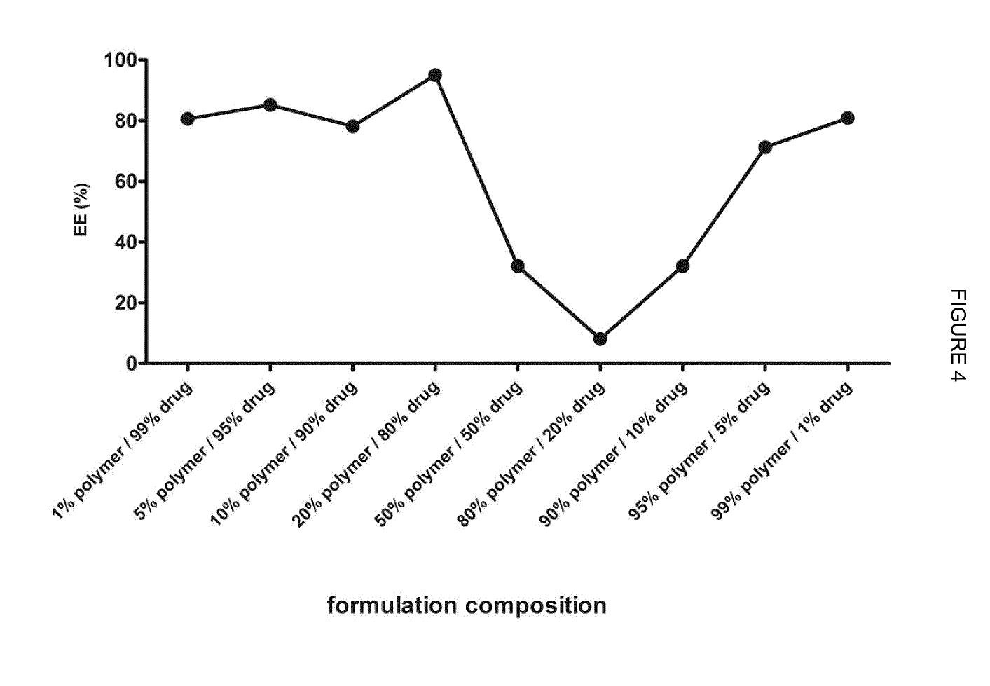

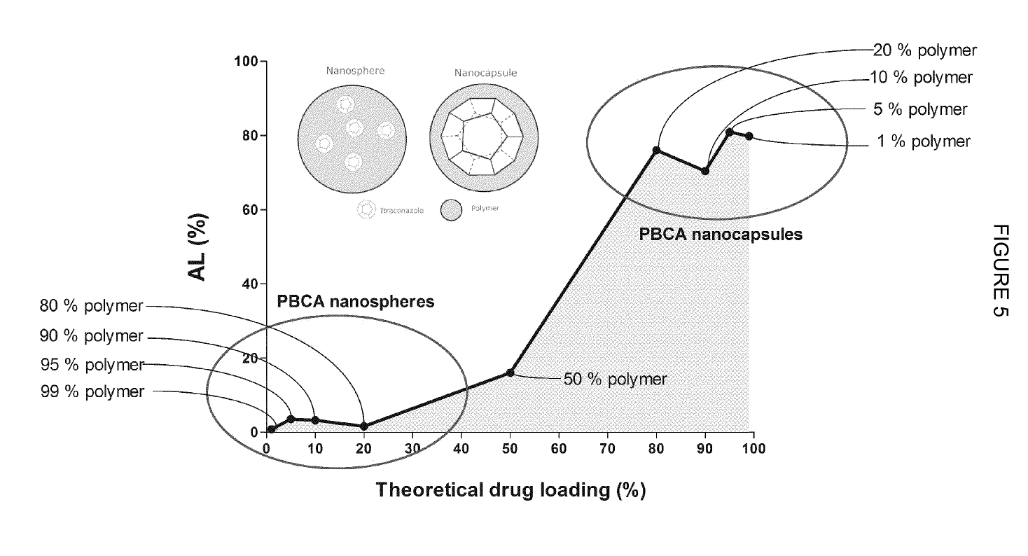

The Itraconazole concentration in the filtered nanoparticle suspensions was determined by Reverse-Phase High Performance Liquid Chromatography (RP-HPLC) in order to calculate the encapsulation efficiency (EE) and absolute drug load (AL). As shown in FIG. 4, high polymer-drug ratios allowed the formation of nanocapsules with high encapsulation efficiency. Moreover, the nanocapsules had significantly higher absolute drug loads than corresponding nanospheres (cf. FIG. 5).

### Example 4 Preparation of Itraconazole-Loaded poly(n-butyl 2-cyanoacrylate) Nanocapsules Using Different Polymer-Drug Ratios

Samples 4#1 to 4#13 were prepared and analyzed as described in EXAMPLE 3, except from using polymer-drug solutions as indicated in Table 2.

TABLE 2
Composition of the solution of Itraconazole
and PBCA in chloroform

| | poly(n-butyl | | |
|---|---|---|---|
| Sample | 2-cyano-acrylate) | Itraconazole | polymer-drug |
| # | [mg/ml CHCl₃] | [mg/ml CHCl₃] | ratio |
| 4#1 | 0.000 | 25.000 | 0:100 |
| 4#2 | 0.025 | 24.975 | 0.1:99.9 |
| 4#3 | 0.250 | 24.750 | 1:99 |
| 4#4 | 1.250 | 23.750 | 5:95 |
| 4#5 | 2.500 | 22.500 | 10:90 |
| 4#6 | 5.000 | 20.000 | 20:80 |
| 4#7 | 12.500 | 17.500 | 50:50 |
| 4#8 | 20.000 | 5.000 | 80:20 |
| 4#9 | 22.500 | 2.500 | 90:10 |
| 4#10 | 23.750 | 1.250 | 95:5 |
| 4#11 | 24.750 | 0.250 | 99:1 |
| 4#12 | 24.975 | 0.025 | 99.9:0.1 |
| 4#13 | 25.000 | 0.000 | 100:0 |

The results of the analyses confirmed the switch between nanocapsules and nanospheres observed in EXAMPLE 3 and demonstrated the amount of drug (relative to the total amount of drug and polymer) could be increased to 99.9%.

### Example 5 Influence of Different Surfactants on the Formation of Highly Drug-Loaded Nanoparticles

Samples 5#1 to 5#32: For each sample, 1 ml of a solution of Itraconazole and poly(n-butyl 2-cyanoacrylate) in chloroform (concentrations as indicated in Table 3) was added to 2 ml of an aqueous solution of 12 μM Tween 80 and a further surfactant (as indicated in Table 3). The resulting mixture was sonicated (70%, 1 cycle) for a time and at a temperature as indicated in Table 3. Then, the chloroform was evaporated at room temperature, and finally the sample was filtered through a 0.2 μm membrane to remove any non-encapsulated Itraconazole (which precipitated in aqueous environment).

TABLE 3
Emulsion solvent evaporation experiments
using different surfactants

| | Solution (I) in | | | | |
|---|---|---|---|---|---|
| | CHCl₃: | | Solution (II) | | |
| Exper- | Itracon- | | in water: 12 μM | Sonication | |
| iment | azole | PBCA | Tween 80 + | time | temper- |
| # | [mg/ml] | [mg/ml] | further surfactant | [min] | ature |
| 5#1 | 10 | 5 | 0.04 mM Lutrol F68* | 10 | ice cooling |
| 5#2 | 50 | 5 | 8.6 mM SDS | 10 | ice cooling |
| 5#3 | 10 | 5 | 10 mM SCh | 10 | RT |
| 5#4 | 50 | 10 | 8.6 mM SDS | 10 | RT |
| 5#5 | 50 | 5 | 1% (wt./vol.) PVA | 4 | ice cooling |
| 5#6 | 50 | 10 | 10 mM SCh | 4 | ice cooling |
| 5#7 | 10 | 10 | 1% (wt./vol.) PVA | 10 | RT |
| 5#8 | 50 | 10 | 1% (wt./vol.) PVA | 10 | ice cooling |
| 5#9 | 50 | 5 | 0.04 mM Lutrol F68* | 10 | RT |
| 5#10 | 10 | 10 | 8.6 mM SDS | 4 | RT |
| 5#11 | 50 | 5 | 8.6 mM SDS | 4 | RT |
| 5#12 | 10 | 10 | 8.6 mM SDS | 10 | ice cooling |
| 5#13 | 10 | 10 | 10 mM SCh | 4 | RT |
| 5#14 | 10 | 5 | 8.6 mM SDS | 4 | ice cooling |
| 5#15 | 10 | 5 | 1% (wt./vol.) PVA | 4 | RT |
| 5#16 | 10 | 5 | 1% (wt./vol.) PVA | 10 | ice cooling |
| 5#17 | 50 | 10 | 0.04 mM Lutrol F68* | 4 | RT |
| 5#18 | 50 | 5 | 0.04 mM Lutrol F68* | 4 | ice cooling |
| 5#19 | 10 | 5 | 8.6 mM SDS | 10 | RT |
| 5#20 | 50 | 10 | 8.6 mM SDS | 4 | ice cooling |
| 5#21 | 50 | 5 | 10 mM SCh | 4 | RT |
| 5#22 | 10 | 5 | 10 mM SCh | 4 | ice cooling |
| 5#23 | 50 | 10 | 1% (wt./vol.) PVA | 4 | RT |
| 5#24 | 10 | 10 | 0.04 mM Lutrol F68* | 4 | ice cooling |
| 5#25 | 10 | 5 | 0.04 mM Lutrol F68* | 4 | RT |
| 5#26 | 50 | 10 | 0.04 mM Lutrol F68* | 10 | ice cooling |
| 5#27 | 10 | 10 | 0.04 mM Lutrol F68* | 10 | RT |
| 5#28 | 50 | 5 | 10 mM SCh | 10 | ice cooling |
| 5#29 | 50 | 10 | 10 mM SCh | 10 | RT |
| 5#30 | 10 | 10 | 10 mM SCh | 10 | ice cooling |
| 5#31 | 50 | 5 | 1% (wt./vol.) PVA | 10 | RT |
| 5#32 | 10 | 10 | 1% (wt./vol.) PVA | 4 | ice cooling |

SDS = sodium dodecyl sulfate;
SCh = sodium cholate;
PVA = poly(vinyl alcohol);
RT = room temperature
*Lutrol F68 = Poloxamer 188 (poly(ethylene glycol)-block-poly(propylene glycol)-block-poly(ethylene glycol))

Analysis by light microscopy showed that only the samples containing sodium cholate (5#3, 5#6, 5#13, 5#21, 5#22, 5#28, 5#29 and 5#30) formed stable and uniform nanoparticle suspensions.

The amount of encapsulated Itraconazole in the filtered sample was measured and the encapsulation efficiency (EE) was calculated (EE=[amount of encapsulated Itraconazole]/[total amount of Itraconazole]). The highest encapsulation efficiencies were found in the preparations of samples containing sodium cholate (5#3, 5#6, 5#13, 5#21, 5#22, 5#28, 5#29 and 5#30).

### Example 6 Nanocapsule Formation in the Absence of Tween 80

Samples 6#1 to 6#8 were prepared as described for 5#3, 5#6, 5#13, 5#21, 5#22, 5#28, 5#29 and 5#30 in EXAMPLE 5, except for omitting Tween 80. PBCA nanocapsules containing itraconazole were successfully produced. Thus, sodium cholate was found to be the surfactant which, in combination with preformed polymer, allows the production of stable highly-drug loaded nanoparticles.

### Example 7 Nanocapsule Formation with a Different Polymer

Experiment 7#1 was performed as described for experiment 5#21, except from using 1 mg/ml poly(ethyl 2-cyanoacrylate) (PECA) instead of 5 mg/ml PBCA and 10 mg/ml instead of 50 mg/ml Itraconazole. The z-average diameter of the resulting nano-capsules was determined using a Zetasizer device as described herein (cf. Table 4).

TABLE 4
Z-average diameter of PBCA and PECA nanocapsules

| Experiment 5#21 | Experiment 7#1 |
|---|---|
| (PBCA nanocapsules) | (PECA nanocapsules) |
| 143 nm | 125 nm |

### Example 8 Nanoparticle Formation with and without a Shell-Forming Polymer

Experiment 8#1 was performed as described for experiment 5#21, except from using Lopinavir instead of Itraconazole.

Experiment 8#2 was performed as described for experiment 5#21, except from omitting the polymer (PBCA) and using Lopinavir instead of Itraconazole.

Experiment 8#3 was performed as described for experiment 5#21, except from omitting the polymer (PBCA).

It was found that drug nanoparticles formed in the presence of sodium cholate, despite the absence of polymer. However, the nanoparticles formed in the absence of shell-forming polymer were larger than the corresponding nanocapsules having a polymeric shell (cf. Table 5).

TABLE 5
Z-average diameter of Itraconazole and Lopinavir nanoparticles

| Experiment 8#1 | Experiment 8#2 | Experiment 8#3 |
|---|---|---|
| (Lopinavir-loaded PBCA | (Lopinavir | (Itraconazole |
| nanocapsules) | nanoparticles) | nanoparticles) |
| 457 nm | 724 nm | 171 nm |

### Example 9 FTIR Analysis of PBCA Nanoparticles

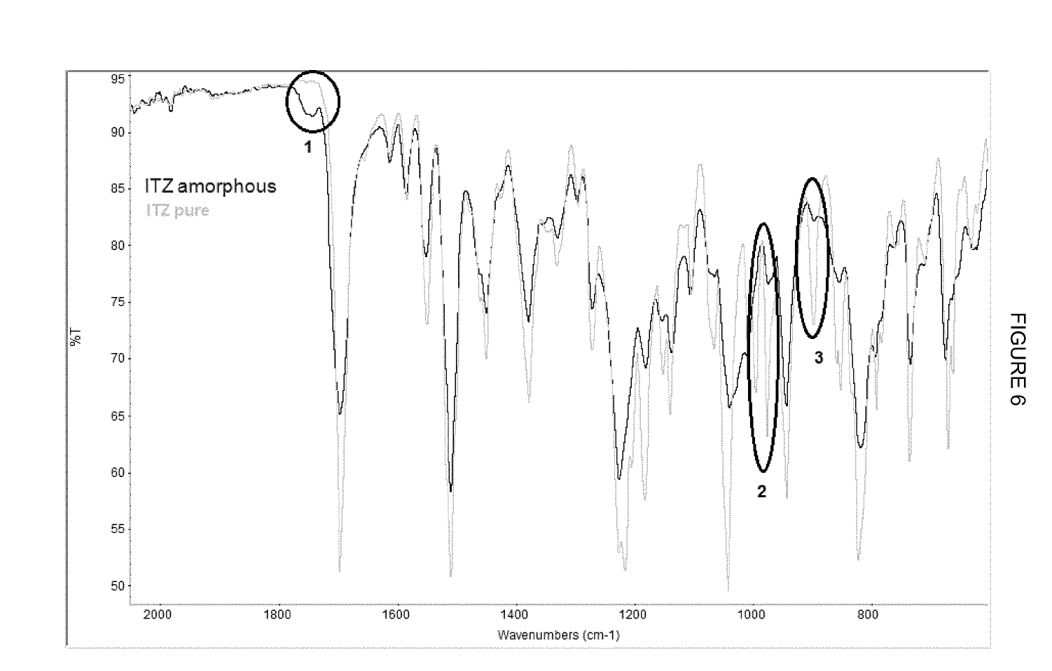

The nanoparticles prepared in EXAMPLE 3 and EXAMPLE 4 were analyzed by Fourier Transform Infrared (FTIR) spectroscopy analysis. For reference purposes, the spectra of amorphous Itraconazole (prepared by exposure to temperatures of >166° C.) and crystalline Itraconazole were measured and compared. It was found that the amorphous Itraconazole is characterized by an FTIR band at approximately 1700-1800 cm⁻¹, while two bands, one at approximately 1000-950 cm⁻¹ and one at approximately 900 cm⁻¹ were indicative of crystalline Itraconazole (cf. FIG. 6). Prior to FTIR analysis, the nanoparticle samples were filtered through a 200 nm membrane to remove any Itraconazole precipitates.

The band at approximately 900 cm⁻¹ was used as an indicator for the (amorphous or crystalline) state of the Itraconazole in the nanoparticles. Said band was detected in the highly drug-loaded PBCA nanocapsules, indicating that the Itraconazole in the nanocapsule core was present in a crystalline state.

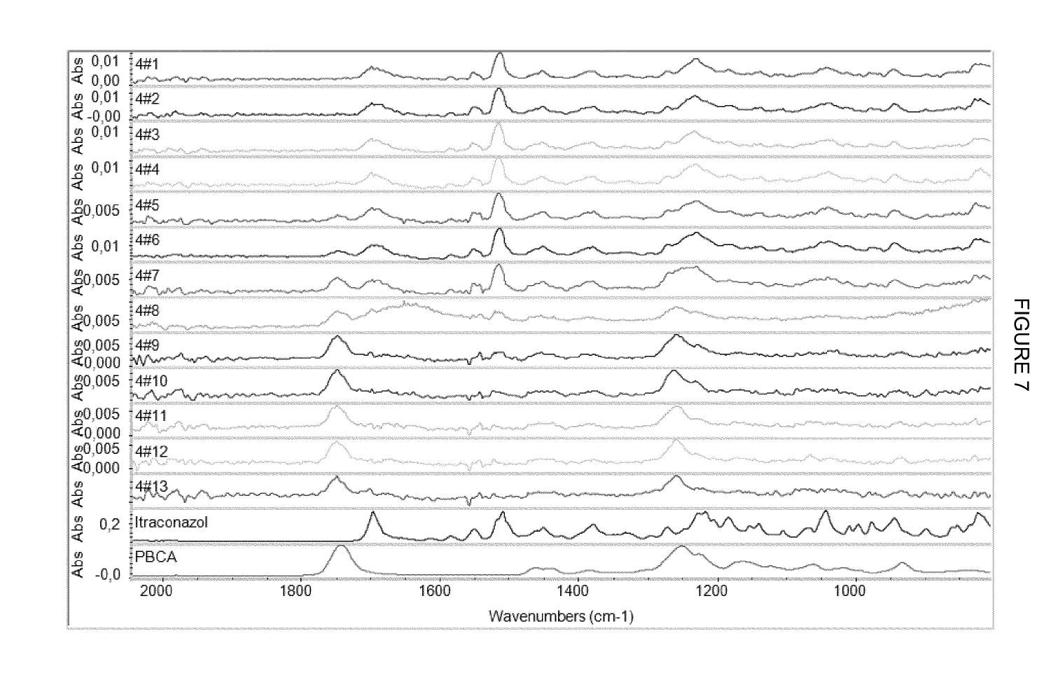

Moreover, specific bands at approximately 1500 cm⁻¹ and 1700 cm⁻¹, which are characteristic for pure crystalline Itraconazole, were clearly detectable in samples 4#1-4#7 (PBCA nanocapsules prepared from polymer-drug ratios of 0:100 to 50:50). In contrast, bands at approximately 1750 cm⁻¹ and 1250 cm⁻¹, which are characteristic for PBCA, were very prominent in samples 4#8 to 4#13 (PBCA nanospheres prepared from polymer-drug ratios of 80:20 to 100:0) (cf. FIG. 7).

### Example 10 Nanocapsule Formation with Different Cargo Molecules

PBCA nanoparticles were prepared and analyzed as described in EXAMPLE 3, except from using Lopinavir (LPV) or the positive allosteric modulator of metabotropic glutamate receptor subgroup 2 (nnGluR2PAM) instead of Itraconazole.

The results confirmed a switch between highly drug-loaded nanocapsules (prepared from polymer-drug ratios of 1:99 to 50:50) and smaller nanospheres having lower drug load (prepared from polymer-drug ratios of 80:20 to 99:1).

### Example 11 Nanocapsule Size Reduction by Addition of Span 80

The z-average diameter of the nnGluR2PAM PBCA nanocapsules prepared in EXAMPLE 10 was about 300 nm. Experiments showed that the addition 0.15 M Span 80 to the solution of PBCA and nnGluR2PAM in chloroform (while keeping the other conditions unchanged) allowed for the preparation of nanocapsules having z-average diameters of only about 90 nm.

### Example 12 Itraconazole-Loaded PBCA Nanoparticles with Shell-Integrated Lipids

Suspensions of nanoparticles (nanocapsules=NC and nanospheres=NS) 12#1 to 12#8 were prepared as follows:

For each sample, the ingredients indicated in one line of Table 6 were combined to obtain a lipophilic phase. Said lipophilic phase was added to 2 ml of an aqueous solution of 10 mM sodium cholate and 12 μM Tween 80 and the sample was sonicated (70%, 1 cycle) for 10 min at room temperature. The chloroform was evaporated by stirring at room temperature (monitored gravimetrically). The Itraconazole-containing nanoparticles were filtered through a 200 nm membrane.

The obtained nanoparticle suspension was purified by concentrating the suspension to about a tenth of its volume using a Vivaspin 500 membrane (300 kDa MWCO, Sartorius, Germany), replenishing the removed suspension medium with fresh aqueous solution of 10 mM sodium cholate and 12 μM Tween 80, and repeating these washing steps for several times to remove any free fluorescent lipids.

The fluorescence intensity of the purified nanoparticle suspension was measured in duplicates. The results indicate a successful fluorescence labeling of the nanoparticles of samples 12#1 to 12#8. The incorporation of the fluorescent lipid did not change the z-average diameter or PDI of the nanoparticles.

TABLE 6
Compositions of the lipophilic phase

| | PBCA | Itraconazole | PEG-FITC | PE-CF |
|---|---|---|---|---|
| | solution | solution | solution | solution |
| | (10 mg/ml | (10 mg/ml | (7.5 mg/ml | (5 mg/ml |
| sample | CHCl₃) | CHCl₃) | CHCl₃) | CHCl₃) |
| # | [μl] | [μl] | [μl] | [μl] |
| 12#1 | 50 | 950 | 10 | — |
| 12#2 | 950 | 50 | 10 | — |
| 12#3 | 1000 | — | 10 | — |
| 12#4 | 50 | 950 | — | 10 |
| 12#5 | 950 | 50 | — | 10 |
| 12#6 | 1000 | — | — | 10 |
| 12#7 | 1000 | — | 80 | — |
| 12#8 | 1000 | — | — | 100 |

PEG-FITC = Methoxyl PEG Fluorescein, MW 40,000 (Nanocs Inc.)
PE-CF = 1,2-dioleyl-sn-glycero-3-phosphoethanolamine-N-(carboxyfluorescein) (ammonium salt) (Avanti Polar Lipids Inc.)

1. A nanocapsule comprising:
a) one or more than one polymer forming a polymeric shell, the polymer(s) comprising a main monomeric constituent selected from one or more than one of C₁-C₁₀-alkyl cyanoacrylates and C₁-C₆-alkoxy-C₁-C₁₀-alkyl cyanoacrylates;
b) one or more than one pharmaceutically or cosmetically active agent comprised in a core encapsulated by said polymeric shell; and
c) a nanoparticle stabilizing agent selected from one or more than one bile acid, one or more than one salt of a bile acid, and mixtures thereof.

2. The nanocapsule of claim 1, wherein the one or more than one active agent (b) is a water-insoluble or poorly water-soluble compound.

3. The nanocapsule of claim 2, wherein the solubility of the one or more than one active agent (b) in water at 25° C. and at pH 7.0 is 0.1 g/100 ml or less.

4. The nanocapsule of claim 1, wherein the one or more than one active agent (b) has a molecular weight in the range of less than 2000 g/mol.

5. The nanocapsule of claim 1, wherein at least 50% of the one or more than one active agent (b) is present in an undissolved solid form.

6. The nanocapsule of claim 1, wherein at least 50% of the one or more than one active agent (b) is present in a crystalline state.

7. The nanocapsule of claim 1, wherein at least 50% of the one or more than one active agent (b) is present in an amorphous state.

8. The nanocapsule of claim 1, wherein at least 50% of the one or more than one active agent (b) is present in a semi-crystalline state.

9. The nanocapsule of claim 1, wherein the main monomeric constituent of the shell-forming polymer(s) (a) is selected from one or more than one of methyl 2-cyanoacrylate, 2-methoxyethyl 2-cyanoacrylate, ethyl 2-cyanoacrylate, n-butyl 2-cyanoacrylate, 2-octyl 2-cyanoacrylate and isobutyl 2-cyanoacrylate.

10. The nanocapsule of claim 9, wherein the one or more than one shell-forming polymer (a) is selected from poly(n-butyl 2-cyanoacrylate), poly(ethyl 2-cyanoacrylate), and mixtures thereof.

11. The nanocapsule of claim 1, wherein the nanoparticle stabilizing agent (c) is a bile acid selected from the group consisting of cholic acid, taurocholic acid, glycocholic acid, deoxycholic acid, lithocholic acid, chenodeoxycholic acid, dehydrocholic acid, ursodeoxycholic acid, hyodeoxycholic acid and hyocholic acid, or a salt of said bile acids, or a mixture of more than one of said bile acids and/or more than one of said bile salts.

12. The nanocapsule of claim 11, wherein the nanoparticle stabilizing agent (c) is selected from one or more than one of cholic acid, salts of cholic acid, and mixtures thereof.

13. The nanocapsule of claim 12, wherein the nanoparticle stabilizing agent (c) is sodium cholate.

14. The nanocapsule of claim 1, wherein the amount of the nanoparticle stabilizing agent (c) is from 3 to 36 wt-% relative to the total weight of shell-forming polymer(s) (a) and active agent(s) (b) of the nanocapsule.

15. The nanocapsule of claim 1, wherein the nanocapsule is basically free of any monomers of the shell-forming polymer(s).

16. The nanocapsule of claim 1, wherein the diameter of the nanocapsule is less than 500 nm.

17. The nanocapsule of claim 16, wherein the diameter of the nanocapsule is in the range of from 50-200 nm.

18. The nanocapsule of claim 1, wherein the amount of the active agent(s) (b) is at least 50 wt-% relative to the total weight of shell-forming polymer(s) (a) and active agent(s) (b) of the nanocapsule.

19. The nanocapsule of claim 1, wherein the amount of the active agent(s) (b) is at least 80 wt-% relative to the total weight of shell-forming polymer(s) (a) and active agent(s) (b) of the nanocapsule.

20. The nanocapsule of claim 1, further comprising one or more than one uptake mediator selected from polyoxyethylene sorbitan fatty acid esters.

21. The nanocapsule of claim 20, wherein the uptake mediator is polyoxyethylene (20) sorbitan monooleate.

22. The nanocapsule of claim 1, further comprising comprises one or more than one sorbitan fatty acid ester.

23. The nanocapsule of claim 22, wherein the sorbitan fatty acid ester is sorbitan monooleate.

24. The nanocapsule of claim 1, further comprising one or more than one amphilic lipids.

25. The nanocapsule of claim 24, wherein the amphilic lipid is selected from the group consisting of naturally occurring or synthetic phospholipids, cholesterols, lysolipids, sphingomyelins, tocopherols, glucolipids, stearylamines and cardiolipins.

26. A plurality of nanocapsules of claim 1 comprising a population of nanocapsules having a diameter of less than 500 nm, wherein the nanocapsules of the population comprise at least 50 wt-% of the active agent(s) (b) relative to the total weight of shell-forming polymer(s) (a) and active agent(s) (b) of the population.

27. The plurality of nanocapsules of claim 26, wherein the population of nanocapsules having a diameter of less than 500 nm accounts for more than 90 wt-% of the plurality of nanocapsules.

28. The plurality of nanocapsules of claim 26 comprising a sub-population of nanocapsules having a diameter in the range of from 50-200 nm, wherein the nanocapsules of the sub-population comprise at least 50 wt-% of the active agent(s) (b) relative to the total weight of shell-forming polymer(s) (a) and active agent(s) (b) of the sub-population.

29. The plurality of nanocapsules of claim 28, wherein the nanocapsules of the subpopulation comprise at least 80 wt-% of the active agent(s) (b) relative to the total weight of shell-forming polymer(s) (a) and active agent(s) (b) of the sub-population.

30. The plurality of nanocapsules of claim 28, wherein the sub-population of nanocapsules having a diameter in the range of from 50-200 nm accounts for more than 90 wt-% of the plurality of nanocapsules.

31. A method for preparing nanocapsules, the method comprising:
i) providing a hydrophobic liquid phase comprising:
one or more than one shell-forming polymer comprising a main monomeric constituent selected from one or more than one of C₁-C₁₀-alkyl cyanoacrylates and C₁-C₆-alkoxy-C₁-C₁₀-alkyl cyanoacrylates, and
one or more than one pharmaceutically or cosmetically active agent dissolved in a non-water-miscible organic solvent or a mixture of two or more non-water-miscible organic solvents;
ii) providing a hydrophilic liquid phase comprising:
a nanoparticle stabilizing agent selected from one or more than one bile acid, or one or more than one salt of a bile acid, or mixtures thereof
dissolved in a hydrophilic solvent;
iii) finely dispersing the hydrophobic liquid phase in the hydrophilic liquid phase so as to form an emulsion; and
iv) removing at least part of the organic solvent(s) from the homogenized mixture so as to obtain a suspension of nanocapsules in the hydrophilic solvent.

32. The method of claim 31, wherein the concentration of the nanoparticle stabilizing agent in the hydrophilic liquid phase provided in step (ii) is in the range of from 50-150% of its critical micelle concentration.

33. The method of claim 31, wherein the hydrophilic liquid phase provided in step (ii) further comprises one or more than one uptake mediator selected from polyoxyethylene sorbitan fatty acid esters.

34. The method of claim 31, wherein the hydrophobic liquid phase provided in step (i) further comprises one or more than one sorbitan fatty acid ester.

35. The method of claim 31, wherein the shell-forming polymer(s), the active agent(s), the nanoparticle stabilizing agent, the uptake mediator and the sorbitan fatty acid ester, respectively, are as defined in claim 2.

36. The method of claim 31, wherein step (iii) is carried out by homogenization under pressure and/or ultrasonically.

37. The method of claim 31, wherein in step (iv) the organic solvent(s) is/are evaporated.

38. A nanocapsule obtainable by the method of claim 31.

39. A pharmaceutical composition comprising a plurality of nanocapsules according to claim 1, and a pharmaceutically acceptable carrier.
